(19) United States
(12) Patent Application Publication
(10) Pub. No.: US 2009/0054959 A1
(43) Pub. Date: Feb. 26, 2009

(54) THERAPEUTIC FOOT AND LEG WARMER

(76) Inventor: CHARLES E. FELKER, Gill, CO (US)

Correspondence Address: Benjamin Hudson Jr., 197 Turman Dr., Fort Collins, CO, 80525, US

(21) Appl. No.: 11/844,857

(22) Filed: Aug. 24, 2007

(51) Int. Cl. A61F 7/00; A61F 7/08; H05B 1/00

(52) U.S. Cl. 607/111; 219/211; 36/2.6

(57) ABSTRACT

There is provided by this invention a foot warming device that is light weight comprising a lower foot portion and an tipper leg portion. The upper leg portion is comprised of flaps that open in the front to allow easy entry of the foot and leg and has hook and loop type connectors manufactured under the trademark Velcro to adjustably fasten to wrap the flaps around the foot and leg to enclose them within the foot warmer. Inserted between an outer covering and the insulating inner lining of the lower foot portion and the upper leg portion are electrically conductive heating elements wherein the resistance of the heating elements is designed so that when the foot warmer is connected to a source of low voltage power such as 120 V it provides sufficient heat to warm the inner insulating limiting to sufficient temperatures for therapeutic purposes.

### BACKGROUND OF THE INVENTION

1. Field of the Invention

This invention relates generally to thermostatically controlled electrically heated footwear, and more particularity, to footwear that provides electric heating to the foot and lower legs for warming and therapeutic purposes.

2. Brief Description of the Prior Art

U.S. Pat. No. 3,621,191 discloses electrically heated footwear and hand wear in which an ionomer plastic insulating layer is fitted into a shoe or other footwear and a mitten or other hand wear. A long stranded heating wire is embedded in a heat-conducting plastic member which is positioned beneath the insole of the footwear and within the insulating plastic cover of the mitten. The wires are connected to a source of low-voltage electricity for resistance heating in order to maintain the footwear and hand wear at a comfortable temperature.

U.S. Pat. No. 3,392,264 discloses electrically heated footwear for cold weather outdoor activities having a heating element that provides heat to the foot of the wearer. Insulated conducting wears are connected to battery for resistive heating.

See also U.S. Pat. No. 5,623,772 which discloses a foot-warming device for a boot that includes a heating element able to be positioned inside the boot, a battery pack for providing power to the heating element, and an electrical cord for detachably connecting electrically the heating element and the battery pack. A support assembly for detachably supporting the battery pack is also provided, which support assembly includes an outer covering that is fastened to the boot's upper surface so as to form a pocket that opens toward the top of the boot for receiving the battery pack. This outer covering is at least partially formed of stretchable material.

U.S. Pat. No. 6,003,248 discloses a liner for footwear, in particular for ski boots, climbing boots or the like, with a thermoplastic and a heating arrangement, and a process for adapting such a liner to the shape of the foot of the person using such a liner. In order to achieve optimum adaptation in a simple manner, it is provided that the electric heating arrangement is formed by at least one heating film 1 which is arranged in the liner and with the aid of which the thermoplastic of the liner can be heated so that it can be adapted to the shape of the foot of the person using the liner.

### SUMMARY OF THE INVENTION

Resistive heating elements are mounted in a moccasin type boot that is connected to an electric circuit. The foot and leg warmer is controlled by a thermostat or other controller connected in the circuit with the resistive heating elements. The front of the boot from the leg section down to the foot is open for easy foot entry. The top of the leg portion wraps around the leg and is connected in the front by hook and loop type material that is manufactured wider the trade name VELCRO.

An embodiment of this invention discloses a foot warmer comprising a bottom portion; an portion that is disposed to receive the leg and foot of a user; and the upper portion is generally composed of two flaps to allow easy entry into the foot warmer and further comprising connectors to adjustably fasten to wrap the flaps around the foot and to enclose them within the foot warmer.

### BRIEF DESCRIPTION OF THE DRAWINGS

FIG. 1 illustrates a front view of a foot and leg warmer in the open position for easy entry incorporating the principles of this invention;

FIG. 2 illustrates a front view of a foot and leg warmer in the closed position incorporating the principles of this invention;

FIG. 3 illustrates a side view of a foot and leg warmer in the closed position incorporating the principles of this invention; and

FIG. 4 illustrates a sectional view of a foot and leg warmer incorporating the principles of this invention.

### BRIEF DESCRIPTION OF THE PREFERRED EMBODIMENT

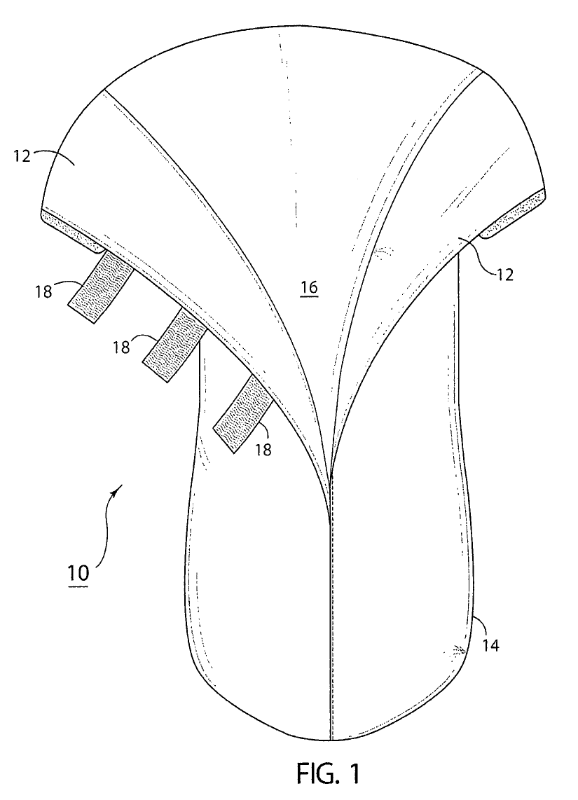

Referring to FIG. 1 a foot warmer 10 incorporating the principles of this invention is shown in the open position where it can be easily seen how the feet and legs are inserted into the foot warmer. FIG. 1 illustrates a foot warmer designed for a person who may have either foot or leg problems where applying heat may be therapeutic treatment or for someone such as a skier, outdoors person, or a person with cold feet who simply wants to keep his feet warm. The invention is may be used by a person sitting or laying down with their bare feet. The front of the boot from the leg section down to the foot is open for easy foot entry. The foot warmer is generally comprised of an upper leg section 12 and a sole section 14. The sole is a soft leather characteristic of a moccasin. The sole section 14 and the upper leg portion are first cut out as one piece and then sewn together in the method of a moccasin. The foot warmer is designed so that it can accommodate any size foot. It is adjustable to avoid placing pressure points on the foot and legs to accommodate swollen feet or legs. The internal inner lining 16 of the foot warmer may be comprised of an insulating fabric such as wool, cotton, insulating plastic foam or other insulating material satisfactory for warmth and comfortable feet. The top of the leg portion wraps around the leg and is connected in the front by hook and loop type material 18 that is manufactured under the trade name VELCRO that is used to provide a secure and snug fit. This invention provides a snug fit when the user's feet and legs are inserted.

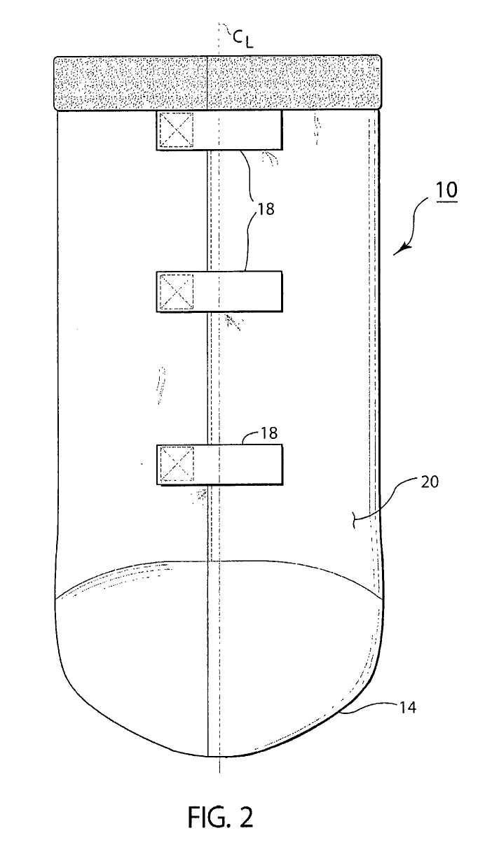

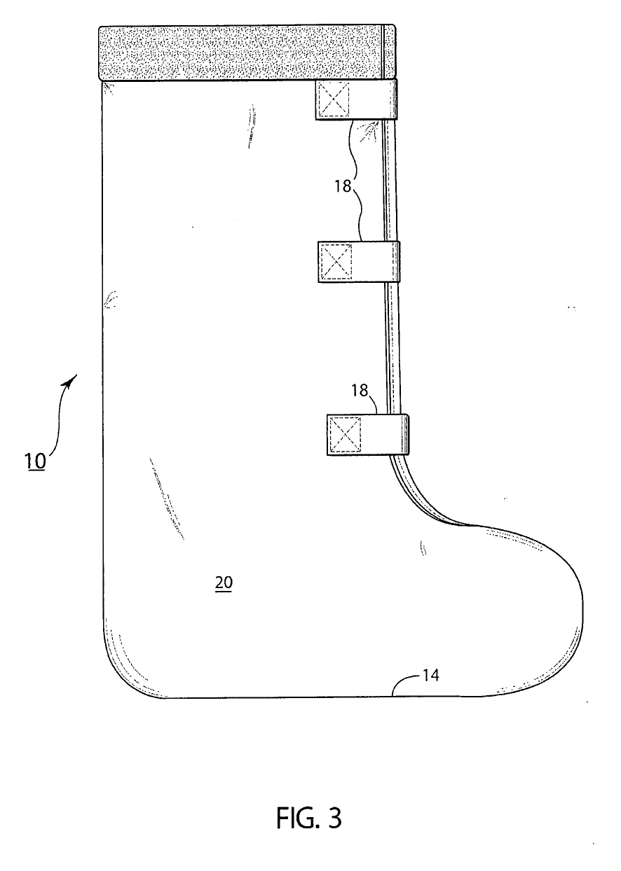

FIG. 2 illustrates the foot warmer in a closed position. As seen in FIG. 2, when the straps 18 are closed, the feet are totally covered and enclosed. The foot warmer is intended for use as a therapeutic boot only and is not intended to be mobile or worn in an outdoors environment. The outer covering 20 of the foot warmer may be comprised of leather, vinyl, cloth or other sturdy material. FIG. 3 illustrates the foot warmer from a side view in the closed position. The height of the foot wanner 10 may extend over the calf portion of the leg as high as the knee.

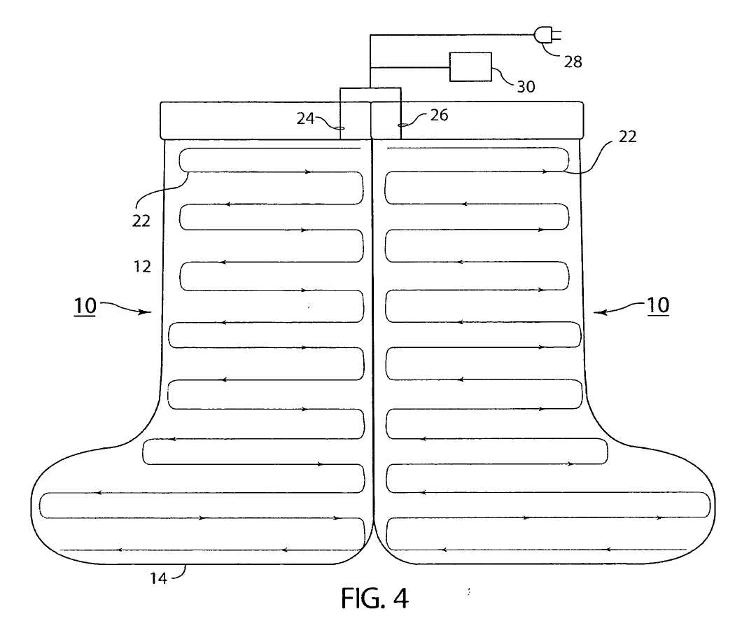

Inserted between the outer covering 20 and the insulating inner lining 16 are electrically conductive heating elements such as electrical resistance heating elements 22 as shown in FIG. 4. The heating elements may be thin resistance elements insulated to prevent the risk of fire and may be woven within the insulating inner lining.

FIG. 4 illustrates two foot warmers one for each foot. The heating elements are wrapped around the foot warmer under the insulating lining from the leg section 12 to the sole 14 and are equally spaced to prevent any hot spots. The resistance of the heating elements is designed so that when the foot warmer is connected to a source of low voltage power such as 120 V it provides sufficient heat to warm the inner insulating lining to sufficient temperatures for therapeutic purposes. Each of the foot warmers has a two conductor wire 24 and 26 that connects to the resistance heater elements 22 to supply 120 volts across the elements in a manner well know to those skilled in the art. An electric plug 28 connects the two conductor wires 24 and 26 to the source of 120 V power by plugging into a convention 120 V outlet A thermostat 30 is connected between the electric plug 28 and the resistance heating elements 22 to control the voltage across the heating elements and thus the current flowing through the elements so that the user can control the heat in the foot warmers 10 to selected temperatures. The thermostat 30 may also be comprised of a timer not shown but well known to those skilled in the art to control the time that power is supplied to the foot warmers 10. The thermostat 30 may also be comprised of a circuit breaker to interrupt power to the foot warmers in the instance of arcing or other electrical failure.

According to the principles of this invention, the foot warmers herein described are simple and easy to manufacture. They provide an electrically powered foot warmer which produces a desirable and controlled heat to the wearer's foot.

The foregoing description of the invention has been presented for purposes of illustration and description. It is not intended to be exhaustive or to limit the invention to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

I claim:

1. A foot warmer comprising:
a lower foot portion;
an upper leg portion extending from the bottom member that is disposed to receive the leg and foot of a user; and
the upper portion being attached to the bottom portion so that it is generally comprised of two flaps to allow easy entry into the foot warmer and further comprising connectors to adjustably fasten to wrap the flaps around the foot and leg to enclose them within the foot warmer.

2. The foot warmer as recited in claim 1, wherein bottom portion and upper portion has attached thereto an internal inner lining comprised of an insulating fabric such as wool, cotton, insulating plastic foam or other insulating material satisfactory for warmth.

3. The foot warmer as recited in claim 2, wherein the upper portion has an outer covering generally comprised of leather, vinyl, cloth or other sturdy material.

4. The foot warmer as recited in claim 1, wherein the flaps are connected in the front by hook and loop type material that is manufactured under the trade name VELCRO.

5. The foot warmer as recited in claim 3, wherein inserted between the outer covering and the insulating inner lining are electrically conductive heating elements.

6. The foot warmer as recited in claim 5, wherein the heating elements may be thin resistance elements insulated to prevent the risk of fire and may be woven within the insulating inner lining.

7. The foot warmer as recited in claim 6, wherein the heating elements are wrapped around the foot warmer under the insulating lining are equally spaced to prevent any hot spots.

8. A foot warmer as recited in claim 1 is designed so that it can accommodate any size foot.

9. The foot warmer as recited in claim 1 wherein the resistance of the heating elements is designed so that when the foot warmer is connected to a source of low voltage power such as 120 V it provides sufficient heat to warm the inner insulating lining to sufficient temperatures for therapeutic purposes.

10. The foot warmer as recited in claim 9 wherein a thermostat is connected between the source of low voltage power and the resistance heating elements to control the voltage across the heating elements and thus the current flowing through the elements so that the user can control the heat in the foot warmers to selected temperatures.

11. The foot warmer in as recited in claim 10 wherein the thermostat may also be comprised of a circuit breaker to interrupt power to the foot warmers in the instance of arcing or other electrical failure.
